(19) United States
(12) Patent Application Publication
(10) Pub. No.: US 2005/0154467 A1
(43) Pub. Date: Jul. 14, 2005

(54) Interconnected spinal device and method

(75) Inventors: Marc M. Peterman, Memphis, TN (US); Steven C. Humphreys, Chattanooga, TN (US); Scott D. Hodges, Ooltewah, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/031,904

(22) Filed: Jan. 7, 2005

Related U.S. Application Data
(60) Provisional application No. 60/534,960, filed on Jan. 9, 2004.

(51) Int. Cl. A61F 2/44

(52) U.S. Cl. 623/17.16; 623/17.15

(57) ABSTRACT

An artificial spinal joint for creating at least a portion of a coupling between a superior vertebra and an inferior vertebra comprises an inferior arthroplasty half. The inferior arthroplasty half comprises an inferior articulating component for placement in an intervertebral disc space between the superior and inferior vertebrae, a first posterior arm, and a first bridge component coupled between the inferior articulating component and the first posterior arm. The artificial spinal joint further includes a superior arthroplasty half. The superior arthroplasty half comprises a superior articulating component for placement in an intervertebral disc space between the superior and inferior vertebrae, a second posterior am, and a second bridge component coupled between the superior articulating component and the second posterior arm. The first posterior arm and the second posterior arm cross an anterior-posterior axis defined centrally through and extending from the intervertebral disc space.

### CROSS-REFERENCE

This application claims priority from U.S. Provisional Patent Application Ser. No. 60/534,960 filed on Jan. 9, 2004, entitled “Posterior Lumbar Arthroplasty.” The following applications also claim priority to the above referenced provisional application and are related to the present application. They are incorporated by reference herein.

U.S. Utility patent application Ser. No. (Attorney Docket No. PC1146), filed on Jan. 7, 2005 and entitled “Spinal Arthroplasty Device and Method;”

U.S. Utility patent application Ser. No. (Attorney Docket No. P21769), filed on Jan. 7, 2005 and entitled “Dual Articulating Spinal Device and Method;”

U.S. Utility patent application Ser. No. (Attorney Docket No. P21756), filed on Jan. 7, 2005 and entitled “Split Spinal Device and Method;”

U.S. Utility patent application Ser. No. (Attorney Docket No. P21745), filed on Jan. 7, 2005 and entitled “Mobile Bearing Spinal Device and Method;”

U.S. Utility patent application Ser. No. (Attorney Docket No. P21743), filed on Jan. 7, 2005 and entitled “Support Structure Device and Method;”

U.S. Utility patent application Ser. No. (Attorney Docket No. P21765), filed on Jan. 7, 2005 and entitled “Centrally Articulating Spinal Device and Method;” and

U.S. Utility patent application Ser. No. (Attorney Docket No. P21751), filed on Jan. 7, 2005 and entitled “Posterior Spinal Device and Method.”

### TECHNICAL FIELD

Embodiments of the invention relate generally to devices and methods for accomplishing spinal surgery, and more particularly in some embodiments, to spinal arthroplasty devices capable of being placed posteriorally into the vertebral disc space. Various implementations of the invention are envisioned, including use in total spine arthroplasty replacing, via a posterior approach, both the disc and facet functions of a natural spinal joint.

### BACKGROUND

As is known the art, in the human anatomy, the spine is a generally flexible column that can take tensile and compressive loads, allows bending motion and provides a place of attachment for ribs, muscles and ligaments. Generally, the spine is divided into three sections: the cervical, the thoracic and the lumbar spine. FIG. 1 illustrates schematically the lumbar spinal 1 and the sacrum regions 3 of a healthy, human spinal column. The sections of the spine are made up of individual bones called vertebrae and the vertebrae are separated by intervertebral discs which are situated therebetween.

FIG. 2 illustrates a portion of the right side of a lumbar spinal region with a healthy intervertebral disc 5 disposed between two adjacent vertebrae 7, 9. In any given joint, the top vertebra may be referred to as the superior vertebra and the bottom one as the inferior vertebra. Each vertebra comprises a generally cylindrical body 7a, 9a, which is the primary area of weight bearing, and three bony processes, e.g., 7b, 7c, 7d (two of which are visible in FIG. 2). As shown in FIG. 7A, in which all of the processes are visible, processes 7b, 7c, 7d extend outwardly from vertebrae body 7 at circumferentially spaced locations. The processes, among other functions, provide areas for muscle and ligament attachment. Neighboring vertebrae may move relative to each other via facet components 7e (FIG. 2), which extend from the cylindrical body of the vertebrae and are adapted to slide one over the other during bending to guide movement of the spine. There are two facet joints, each defined by upper and lower facet components, associated with adjacent vertebra. A healthy intervertebral disc is shown in FIG. 3. As shown in FIG. 3, an intervertebral disc has 4 regions: a nucleus pulposus 11, a transition zone 13, an inner annulus fibrosis region 15 and an outer annulus fibrosis 17. Generally, the inner annulus fibrosis region 15 and the outer annulus fibrosis region 17 are made up of layers of a fibrous gristly material firmly attached to the vertebral bodies above and below it. The nucleus pulposus 11 is typically more hydrated in nature.

These intervertebral discs function as shock absorbers and as joints. They are designed to absorb the compressive and tensile loads to which the spinal column may be subjected while at the same time allowing adjacent vertebral bodies to move relative to each other a limited amount, particularly during bending (flexure) of the spine. Thus, the intervertebral discs are under constant muscular and/or gravitational pressure and generally are the first parts of the lumbar spine to show signs of “wear and tear”.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis (i.e., spine fusion), which has been performed both anteriorally and/or posteriorally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be advantageous. It is also known to surgically replace a degenerative disc or facet joint with an artificial disc or an artificial facet joint, respectively. However, none of the known devices or methods provide the advantages of the embodiments of the present disclosure.

Accordingly, the foregoing shows there is a need for an improved spinal arthroplasty that avoids the drawbacks and disadvantages of the known implants and surgical techniques.

### SUMMARY

In one embodiment, an artificial spinal joint for creating at least a portion of a coupling between a superior vertebra and an inferior vertebra comprises an inferior arthroplasty half. The inferior arthroplasty half comprises an inferior articulating component for placement in an intervertebral disc space between the superior and inferior vertebrae, a first posterior arm, and a first bridge component coupled between the inferior articulating component and the first posterior arm. The artificial spinal joint further includes a superior arthroplasty half. The superior arthroplasty half comprises a superior articulating component for placement in an intervertebral disc space between the superior and inferior vertebrae, a second posterior am, and a second bridge component coupled between the superior articulating component and the second posterior arm. The first posterior arm and the second posterior arm cross an anterior-posterior axis defined centrally through and extending from the intervertebral disc space.

In another embodiment, a method of implanting an artificial spinal joint between superior and inferior vertebrae comprises creating a first exposure through a patient's back to access an intervertebral space and creating a second exposure through the patient's back to access the intervertebral space. The method further comprises delivering a first articulating portion of the artificial spinal joint to the intervertebral space along a first path through the first exposure and delivering a second articulating portion of the artificial spinal joint to the intervertebral space along a second path through the second exposure. The method further comprises engaging the first and second articulating assembly portions to form an intervertebral joint centered about an anterior-posterior axis defined through and extending from the center of the intervertebral disc space and positioning first and second posterior arms of the artificial spinal joint outside of the intervertebral space and across the anterior-posterior axis.

A system for creating a coupling between a superior vertebra and an inferior vertebra, the system comprises rostral and caudal anterior articulating components pivotally engaged about a center of rotation, wherein the rostral and caudal anterior articulating components and the center of rotation are adapted for location within an intervertebral disc space between the superior and inferior vertebrae. The system further comprises rostral and caudal posterior arms adapted for positioning outside of the intervertebral disc space and slidably engaged to move about the center of rotation. The rostral anterior articulating component is adapted for implantation through a first approach into the intervertebral disc space and the caudal articulating component is adapted for implantation through a contralateral approach into the intervertebral disc space.

The embodiments disclosed may be useful for degenerative changes of the lumbar spine, post-traumatic, discogenic, facet pain or spondylolisthesis, and/or to maintain motion in multiple levels of the lumbar spine.

Additional and alternative features, advantages, uses and embodiments are set forth in or will be apparent from the following description, drawings, and claims.

### BRIEF DESCRIPTION OF THE DRAWINGS

FIG. 1 is a side elevation schematic view of the lumbar spinal and the sacrum regions of a healthy, human spinal column.

FIG. 2 is a detailed perspective view showing a portion of the right side of the lumbar vertebrae shown in FIG. 1 with a healthy disc disposed between two vertebrae.

FIG. 3 is a top perspective view of the intervertebral disc shown in FIG. 2 illustrating the major portions of the disc.

FIG. 4 is a side exploded elevation view of a portion of a lumbar spine showing a first embodiment of an artificial intervertebral joint constructed according to the principles of the disclosure.

FIG. 5 is an anterior elevation view of a portion of a lumbar spine showing the superior, disc and inferior portions of the left and right halves of an assembled artificial intervertebral joint constructed according to the first embodiment of the disclosure.

FIG. 6 is a side elevation view of the right half of the artificial intervertebral joint shown in FIG. 5.

FIG. 7A is a transverse, bottom-up-view of a portion of a lumbar spine showing the superior portion of the artificial intervertebral joint illustrated in FIG. 4.

FIG. 7B is a transverse, top-down-view of a portion of a lumbar spine showing the inferior portion of the artificial intervertebral joint illustrated in FIG. 4.

FIG. 8 is a transverse, bottom-up-view of a portion of a lumbar spine showing a second embodiment of a superior portion of an artificial intervertebral joint in which pedicle screws are used to assist in implantation.

FIG. 9 is a transverse, top-down-view of a portion of a lumbar spine showing a second embodiment of an inferior portion of an artificial intervertebral joint in which pedicle screws are used to assist in implantation.

FIG. 10 is a lateral view of a portion of a lumbar spine showing the superior portion of the artificial intervertebral joint shown in FIG. 8 with one of the pedicle screws being visible.

FIG. 11 is a lateral view of a portion of a lumbar spine showing the inferior and integrated disc portions of an artificial integral intervertebral joint shown in FIG. 9 with one of the pedicle screws being visible.

FIG. 12 is a posterior view of a portion of a lumbar spine showing the superior portion of the artificial intervertebral joint shown in FIG. 8 with two pedicle screws being visible.

FIG. 13 is a posterior view of a portion of a lumbar spine showing the inferior portion of the artificial intervertebral joint shown in FIG. 9 with two pedicle screws being visible.

FIG. 14 is a side elevation view of a portion of a lumbar spine showing the second embodiment with pedicle screws in an assembled position.

FIG. 15 is a posterior view of a portion of a lumbar spine showing a third embodiment of the inferior, disc and superior portions of an artificial intervertebral joint in which tension bands are used.

FIG. 16 is a side elevation view of a portion of a lumbar spine showing the third embodiment in which tension bands are used in an assembled position.

FIG. 17 is a transverse, bottom-up-view of a portion of a lumbar spine showing the superior portion of a fourth embodiment of an artificial intervertebral joint constructed according to the principles of the disclosure in which the facet joints are not replaced.

FIG. 18 is a transverse, top-down-view of a portion of a lumbar spine showing the inferior portion of the fourth embodiment of an artificial intervertebral joint.

FIG. 19 is a side view of another embodiment of the present disclosure.

FIG. 20 is an opposite side view of the embodiment of FIG. 19.

FIG. 21 is a posterior perspective view of the embodiment of FIG. 19.

FIG. 22 is an environmental view of the embodiment of FIG. 19.

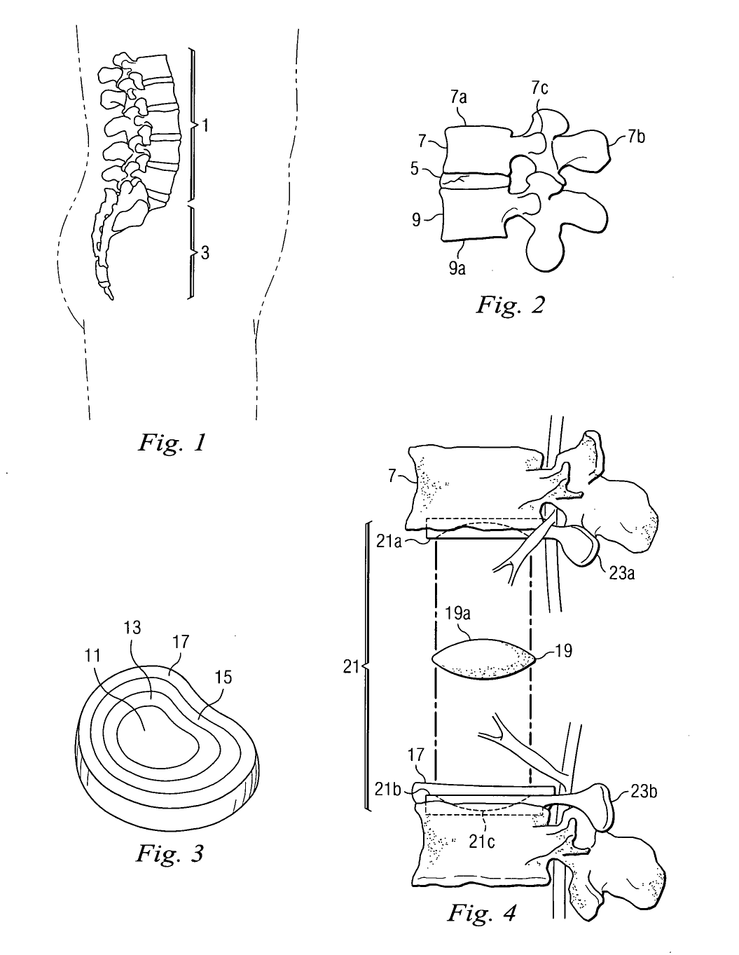

### DESCRIPTION

The drawings illustrate various embodiments of an artificial intervertebral joint for replacing an intervertebral disc or the combination of an intervertebral disc and at least one corresponding facet joint. Various embodiments of the artificial intervertebral joint according to the principles of the disclosure may be used for treating any of the problems that lend themselves to joint replacement including particularly, for example, degenerative changes of the lumbar spine, post-traumatic, discogenic, facet pain or spondylolisthesis and/or to maintain motion in multiple levels of the lumbar spine.

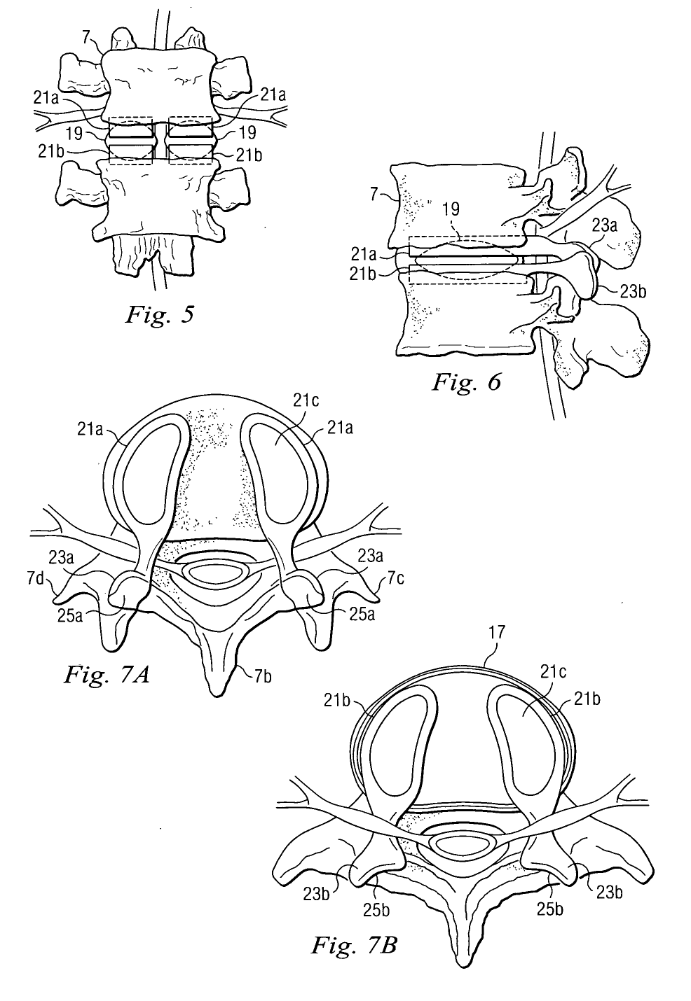

FIGS. 4-7 illustrate a first exemplary embodiment of an artificial intervertebral joint. As illustrated in FIGS. 4 and 5, each joint is composed of two arthroplasty halves, each of which has a spacer or disc 19 and a retaining portion 21. The retaining portion 21 includes a first retaining portion 21a and a second retaining portion 21b. In the example illustrated in FIG. 4, the first retaining portion 21a is superior to (above) the second retaining portion 21b and the disc 19 is situated therebetween. Although the artificial intervertebral joint according to this exemplary embodiment has two halves for each of the first retaining portion and the second retaining portion, it should be understood that alternative embodiments may be implemented such that the artificial intervertebral joint has a single first retaining member, a single second retaining member and a single spacer. It should also be understood that alternative embodiments may also be carried out with arthroplasties having a first retaining portion, a second retaining portion, and/or a disc which each consist of unequal sized halves or more than two components.

Further, as illustrated in FIG. 4, the first retaining portion 21a and the second retaining portion 21b are situated between two adjacent vertebrae. More particularly, the first retaining portion may be situated along an inferior surface of the upper of the two adjacent vertebrae and the second retaining portion may be situated above a superior surface of the lower of the two adjacent vertebrae. However, it should be understood by one of ordinary skill in the art that the first retaining portion and second retaining portion are not limited to such an arrangement, and may be oriented in different positions and/or shaped differently than what is illustrated herein.

The surfaces of the retaining portions 21a, 21b of the arthroplasty that contact the remaining end plates of the vertebrae may be coated with a beaded material or plasma sprayed to promote bony ingrowth and a firm connection therebetween. In particular, the surface to promote bone ingrowth may be a cobalt chromium molybdenum alloy with a titanium/calcium/phosphate double coating, a mesh surface, or any other effective surface finish. Alternatively or in combination, an adhesive or cement such as polymethylmethacrylate (PMMA) may be used to fix all or a portion of the implants to one or both of the endplates.

As discussed in more detail below, a significant portion of the outer annulus region 17 (see, e.g., FIGS. 4, 7B), in some embodiments about 300 degrees, may be retained on the inferior portion of the end plate, which acts as a stop retaining the lower retaining portions in place until bone ingrowth occurs to firmly attach the retaining portions to their respective vertebrae (FIG. 4 only shows a portion of the outer annulus 17 that is retained). In contrast, in conventional anterior arthroplasty about 270 degrees of the outer annulus region 17 typically is removed. In addition, pedicle screws may also be used for immediate fixation as described in more detail in connection with other embodiments discussed below.

In the various embodiments of this disclosure, the first retaining portion 21a and the second retaining portion 21b are structured so as to retain the disc 19 therebetween. For example, in the case of a disc 19 with two convex surfaces 19a, each of the first retaining portion 21a and the second retaining portion 21b may have a concave surface 21c which defines a space within which the disc 19 may be retained. For example, in the exemplary embodiment shown in FIG. 4, the upper convex surface 19a of the disc 19 fits within the concavity defined by the concave surface 21c of the first retaining portion 21a and the lower convex surface 19b of the disc 19 fits within the concavity defined by the concave surface 21c of the second retaining portion 21b.

FIG. 5 illustrates an anterior view of an exemplary assembled artificial intervertebral joint with both arthroplasty halves in place, and FIG. 6 shows a side view of the assembled artificial intervertebral joint shown in FIG. 5. As illustrated in FIGS. 5 and 6, the disc 19 is retained between the first retaining portion 21a and the second retaining portion 21b. It should be understood that although the disc 19 may be held between the first retaining portion 21a and the second retaining portion 21b, the disc 19 is free to slidably move within the space defined by the corresponding surfaces 21a of the first retaining portion 21a and the second retaining portion 21b. In this manner, limited movement between the adjacent vertebrae is provided.

In the exemplary embodiment illustrated in FIGS. 4, 5 and 6, the disc 19 is a separate component which is inserted between the first retaining portion 21a and the second retaining portion 21b. However, as discussed below, it should be understood that the spacer or disc 19 may be integrally formed with or integrated into in one or both of the first retaining portion 21a and the second retaining portion 21b.

In the exemplary embodiment of the disclosure, as illustrated best in FIGS. 4, 6, 7A and 7B, each of the retaining portions of the artificial intervertebral joint includes a first artificial facet component 23a and a second artificial facet component 23b. As shown in FIGS. 7A and 7B, the first artificial facet component 23a has a face 25a and the corresponding second artificial facet component 23b has a face 25b configured such that the face 25a matingly fits with the face 25b to stabilize adjacent vertebrae while preserving and guiding the mobility of each vertebrae with respect to the other vertebrae. Each set of the upper and lower retaining portions 21a, 21b may have a pair of facet components 23a, 23b, which together define a facet joint. For a total joint replacement with facets according to this embodiment, the left and right arthroplasties would define two adjacent facet joints when viewed from the posterior.

Regardless of whether artificial facet joints are provided, the respective upper and lower retaining portions associated with the left and right halves of the arthroplasty may be completely independent from the other. That is, as shown in FIG. 7A, for example, the first retaining portions 21a associated with each half are not in direct contact with each other. The same is true with respect to the second retaining portions 21b shown in FIG. 7B. However, it should be understood by one of ordinary skill in the art that, even in the embodiment of the disclosure which includes artificial facet joints, at least a portion of the first retaining portions 21a of each half and/or at least a portion of the second retaining portions 21b of each half may directly contact and/or be connected to each other as described in more detail in connection with the discussion of FIGS. 17-18.

Further, in the various embodiments of the disclosure, the disc 19, the first retaining portion 21a and the second retaining portion 21b may be made of any appropriate material which will facilitate a connection that transmits compressive and tensile forces while providing for the aforementioned slidable motion in a generally transverse direction between each of the adjacent surfaces. For example, in the first embodiment, the first retaining portion 21a and the second retaining portion 21b may be typically made from any metal or metal alloy suitable for surgical implants such as stainless steel, titanium, and cobalt chromium, or composite materials such as carbon fiber, or a plastic material such as polyetheretherketone (PEEK) or any other suitable materials. The disc may be made from plastic such as high molecular weight polyethylene or PEEK, or from ceramics, metal, and natural or synthetic fibers such as, but not limited to, carbon fiber, rubber, or other suitable materials. Generally, to help maintain the sliding characteristic of the surfaces, the surfaces may be polished and/or coated to provide smooth surfaces. For example, if the surfaces are made of metal, the metal surfaces may be polished metal.

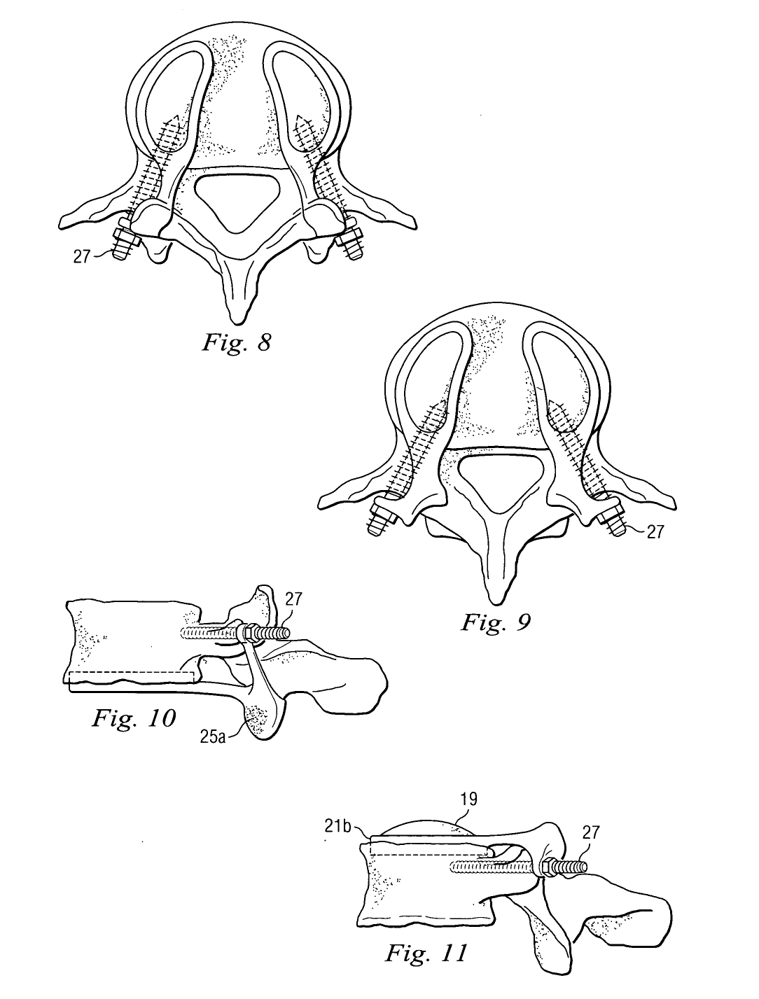

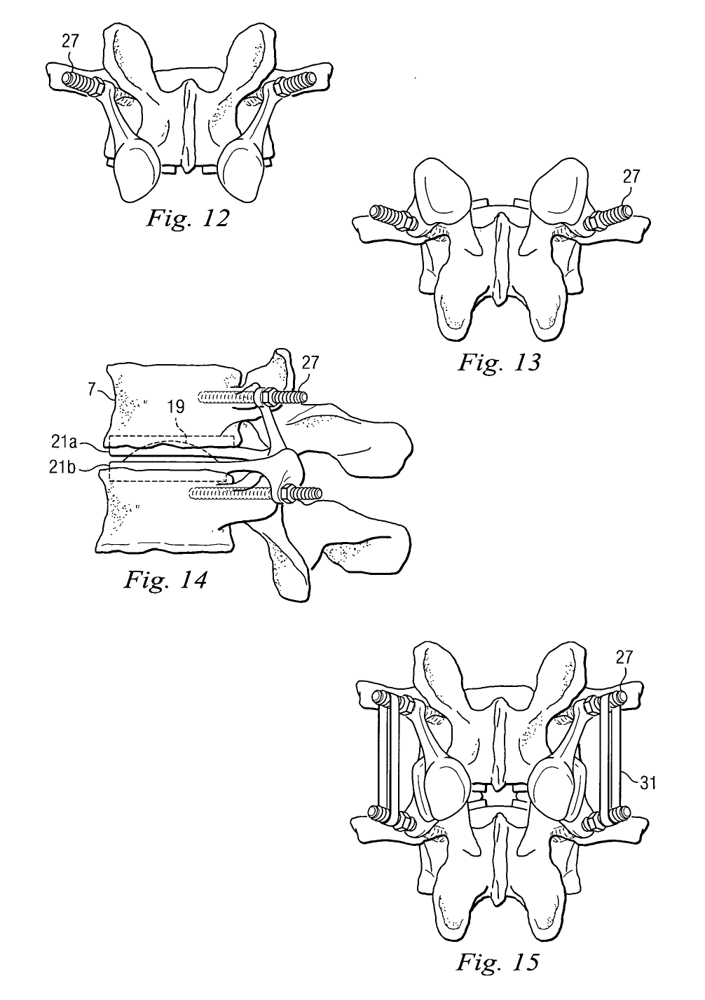

FIGS. 8-14 illustrate a second embodiment of an artificial intervertebral joint. Only features that differ from the first embodiment are discussed in detail herein. In the second exemplary embodiment, securing components, such as, for example, pedicle screws 27 are provided to provide a more secure and immediate connection between each of the first retaining portion 21a and/or the second retaining portion 21b to the corresponding vertebra. In addition, this embodiment illustrates a disc 19 which is integrated with one of the retaining portions, here lower retaining portion 21b. Disc 19 may be integrally formed from the same material as its retaining portion, but also may be separately formed from similar or dissimilar materials and permanently connected thereto to form an integral unit. In this embodiment, the disc 19 and the retaining portions may be all formed from metal.

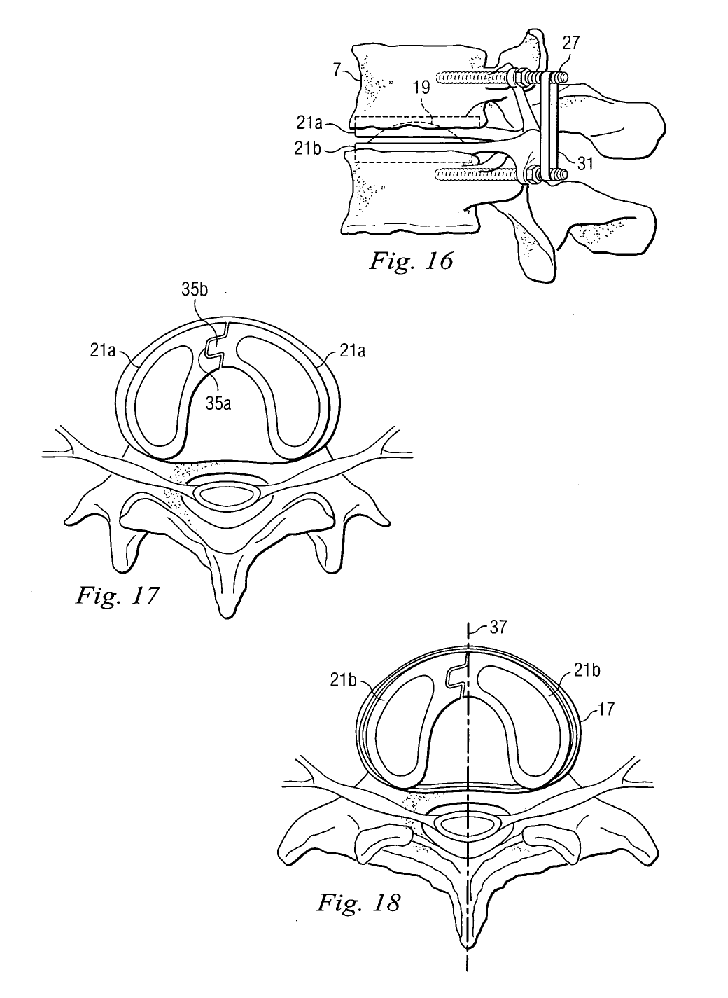

FIGS. 15 and 16 illustrate a third embodiment of an artificial intervertebral joint. In the third exemplary embodiment, additional securing components, such as, for example, tension bands 31 are provided to supplement or replace the function of posterior ligaments that limit the mobility between adjacent vertebrae by securing the first retaining portion 21a to the second retaining portion 21b. As shown in FIGS. 15-16, posterior tension bands 31 may be provided by wrapping them around the corresponding pedicle screws 27 or other convenient attachment points.

FIGS. 17 and 18 illustrate a fourth embodiment of an artificial intervertebral joint. In the exemplary embodiment illustrated in FIGS. 17 and 18, the artificial intervertebral joint may have all of the features discussed above except for artificial facet components. In this embodiment, the natural facet joints remain. The ligamentous tension band may also be left intact in some embodiments. In addition, this embodiment includes a specific example of an anterior midline connection between respective upper and lower retaining portions, which assists in maintaining the placement of the first retaining portion 21a and the second retaining portion 21b.

FIGS. 17 and 18 illustrate that it is possible to provide a first retaining portion 21a with a lock and key type pattern which is complemented by the corresponding mating portion provided on the second retaining portion 21b. More particularly, one half of the first retaining portion 21a has an outer boundary with a U-shaped portion 35a while the other half of the corresponding first retaining portion 21a has an outer boundary with a protruding portion 35b, which fits into the U-shaped portion 35a. As a result, each half of the first retaining portion 21a, 21b may be maintained in a predetermined position. However, the upper or lower retaining portions may fit together and/or be connected in the interbody space, e.g., near their midline anterior portions, in any manner that facilitates implantation and/or assists in providing and/or retaining the joint in a generally stable, symmetrical configuration. It may be even more important to provide such connection between the lower retaining portions due to the inward forces provided by annulus 17 remaining on the inferior end plate as shown in FIG. 18. A midline connection between the respective lower retaining portions will resist the force of the outer annulus tending to cause migration of the retaining portions toward the midline 37.

As shown in the various exemplary embodiments, other than the portions of the first and/or second retaining portions which may fit together like a lock and key to maintain the placement of the portions relative to each other, each half of the artificial intervertebral joint may be generally symmetrical about the midline 37 of the vertebrae.

Again, these exemplary embodiments are merely illustrative and are not meant to be an exhaustive list of all possible designs, implementations, modifications, and uses of the invention. Moreover, features described in connection with one embodiment of the disclosure may be used in conjunction with other embodiments, even if not explicitly stated above.

While it should be readily apparent to a skilled artisan from the discussion above, a brief description of a suitable surgical procedure that may be used to implant the artificial joint is provided below. Generally, as discussed above, the artificial intervertebral joint may be implanted into a body using a posterior transforaminal approach similar to the known TLIF or PLIF procedures. According to this approach, an incision, such as a midline incision, may be made in the patient's back and some or all of the affected disc and surrounding tissue may be removed via the foramina. Depending on whether any of the facet joints are being replaced, the natural facet joints may be trimmed to make room for the artificial facet joints. Then, the halves of the artificial intervertebral joint may be inserted piecewise through the left and right transforaminal openings, respectively. That is, the pieces of the artificial intervertebral joint including the upper and lower retaining portions, with or without facet components, and the artificial disc, if provided separately, fit through the foramina and are placed in the appropriate intervertebral space. The pieces of the artificial joint may be completely separated or two or more of them may be tied or packaged together prior to insertion through the foramina by cloth or other materials known in the art. In cases where at least a portion of the outer annulus of the natural disc can be retained, the lower retaining portions of each side of the artificial intervertebral joint are inserted such that they abut a corresponding portion of the annulus. If a midline anterior connection is provided, the left and right halves of the retaining members are fitted together and held in place by the outer annulus. As such, the remaining portion of the annulus may be in substantially the same place as it was prior to the procedure.

Further, in the cases where the annulus of the natural disc must be removed completely or this is insufficient annulus remaining, it is possible, for example, to use the embodiment of the disclosure where the pedicle screws are implemented so as to be assured that the pieces of the artificial intervertebral joint remain in place. It should be understood by one of ordinary skill in the art that the artificial joint could be implanted via an anterior approach or a combined anterior and posterior approach, although the advantages of a posterior procedure would be limited. For example, some of the pieces of the artificial intervertebral joint may be inserted from an anterior approach and others posteriorly. The anteriorly and posteriorly placed portions could be fitted together similar to the embodiment shown in FIGS. 17 and 18.

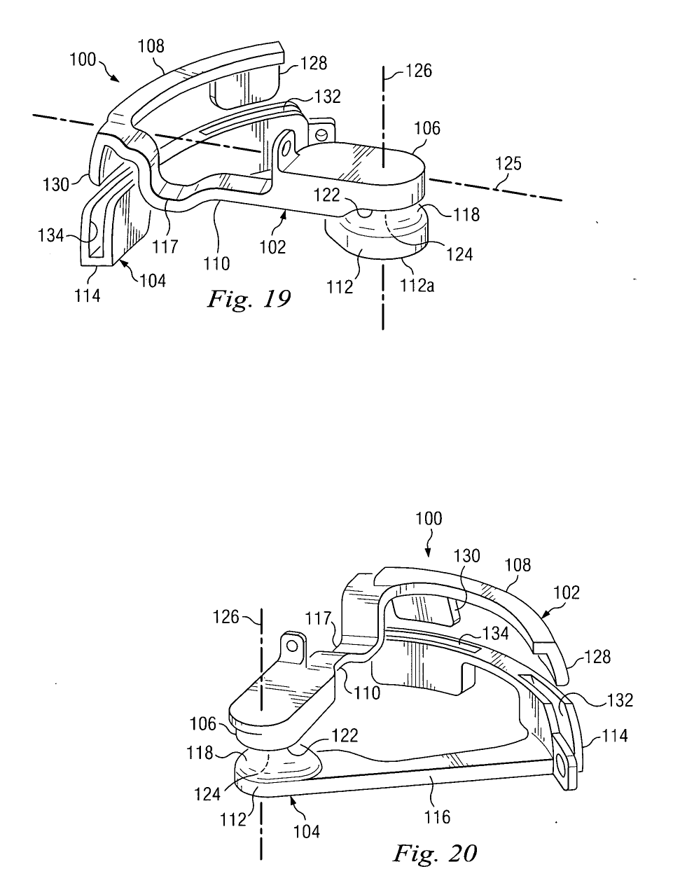

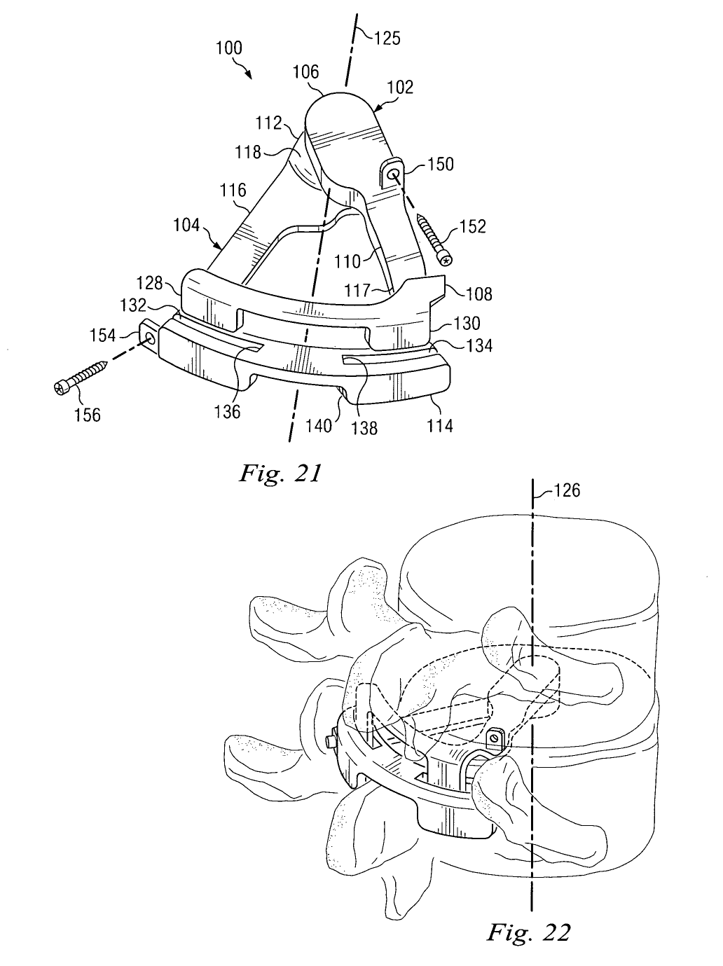

Referring now to FIGS. 19-22, in this embodiment, an artificial intervertebral joint 100 may include two arthroplasty halves 102, 104 which may be inserted between the vertebrae 7, 9. The arthroplasty half 102 may be a superior arthroplasty half and may include a rostral anterior component 106, a rostral posterior joint component 108, and a rostral bridge 110 extending between the anterior component 106 and the posterior component 108. The rostral anterior component 106 may include a bone contacting surface 106a. In this embodiment, the rostral bridge 110 may include a jog 117 to create an exit portal and an artificial foramen for the exiting nerve root.

The terms “rostral” and “caudal” are used in some embodiments to describe the position of components of the embodiments. While rostral is typically used in the art to describe positions toward the head and caudal is used to describe positions toward the tail or foot, as used herein, rostral and caudal are used simply as modifiers for the relative locations of components of the illustrated embodiments. For example, rostral components may be on one side of an illustrated joint, and caudal may be on another side of the joint. Components labeled as rostral or caudal to describe an illustrated embodiment are not intended to limit the orientation of a device or application of a method relative to a patient's anatomy, or to limit the scope of claims to any device or method.

The arthroplasty half 104 may be an inferior arthroplasty half and may include a caudal anterior component 112, a caudal posterior joint component 114, and a caudal bridge 116 extending between the anterior component 112 and the posterior component 114. The caudal anterior component 112 may include a bone contacting surface 112a. Either of the bridges 110, 116, but particularly the caudal bridge 116, may be a “super” or artificial pedicle which may supplement or replace a natural pedicle.

The caudal anterior component 112 may include a caudal articulating surface such as a curved protrusion 118. The rostral anterior joint component 106 may include a rostral articulating surface such as an anterior socket 122 configured to receive the curved protrusion 118. A radius of curvature for the curved protrusion 118 may closely match the radius of curvature for the anterior socket 122 to create a highly constrained ball and socket type engagement. The curved protrusion 118 may pivot within the anterior socket 122 about a center of rotation 124 located on an axis 126 extending through the generally cylindrical bodies 7a, 9a. The center of rotation 124 may also be located on an anterior-posterior axis 125 defined through the center of the intervertebral disc space. In an alternative embodiment, by increasing the radius of curvature for the socket relative to the radius of the curved protrusion, the curved protrusion may be permitted to translate within the socket.

The rostral posterior component 108 may be a posterior arm with a curved tab 128 and a curved tab 130. The curved tabs 128, 130 may have a center of curvature located at the center of rotation 124. The caudal posterior component 114 may include a curved slot 132 and a curved slot 134. The curved slots 132, 134 may also have a center of curvature located at the center of rotation 124. The caudal posterior joint component 114 may further include a limiting wall 136, a limiting wall 138, and a recess or notch 140.

The size and shape of the anterior components 106, 112 and the bridge components 110, 116 may be limited by the constraints of a posterior or transforaminal surgical approach. For example, the anterior components 106, 112 may be configured to cover a maximum vertebral endplate area to dissipate loads and reduce subsidence while still fitting through the posterior surgical exposure, Kambin's triangle, and other neural elements. To achieve the maximum endplate coverage, the anterior components 106, 112 may each include surfaces (not shown) that extend anteriorly from the anterior socket 122 and the curved protrusion 118, respectively. The width of the bridge components 110, 116 may also be minimized to pass through Kambin's triangle and to co-exist with the neural elements.

The arthroplasty halves 102, 104 may further includes fixation features for securing the artificial intervertebral joint 100 to the vertebrae 7, 9. It is understood, however, that in an alternative embodiment, the fixation features may be eliminated. Beyond those described below, the arthroplasty halves 102, 104 may include additional fixation features (not shown) to further secure the artificial intervertebral joint 100 to the adjacent vertebrae or to provide symmetrical fastening. In this embodiment, the superior arthroplasty half 102 may include a connection component 150 extending rostrally from the rostral anterior component 106. The connection component 150 in this embodiment includes an aperture adapted to receive a bone fastener such as a screw 152. The orientation of the connection component 150 permits interbody fixation of the screw 152 to the cylindrical vertebral body 7a.

Arthroplasty half 104 may include a connection component 154 attached to or integrally formed with the caudal posterior component 114. The connection component 154 in this embodiment includes an aperture adapted to receive a bone fastener such as a screw 156. The orientation of the connection component 154 permits the screw 156 to become inserted extrapedicularly such that the screw travels a path angled or skewed away from a central axis defined through a pedicle. In this embodiment, the screw may pass through a wall of the pedicle and may achieve strong cortical fixation. Extrapedicular fixation may be any fixation into the pedicle that does not follow a path down an axis defined generally posterior-anterior through the pedicle. The bone fasteners 152, 156 may be recessed so as not to interfere with articulations, soft tissues, and neural structures.

In an alternative embodiment, for example as shown in FIG. 14, a connection component extending from the posterior component 114 may be oriented to permit the screw to become inserted intrapedicularly such that the screw travels a path generally along the central axis through the pedicle. In still another alternative embodiment, the posterior connection component may connect to the generally cylindrical body portion 9a. It is understood that in other alternative embodiments, the connection components may extend at a variety of angles, in a variety of directions from the various components of the arthroplasty half. For example, a connection component may extend from the rostral bridge rather than the rostral anterior joint component.

As shown in FIGS. 19-22, the rostral components 106, 108, 110 of the superior arthroplasty half 102 are integrally formed with rigid connections between the components. It is understood that in a modular alternative embodiment, these components may be removably coupled to one another. For example, the rostral anterior joint component may be installed separate from the bridge. After the anterior component is in place, the bridge may be attached to the anterior component by any fastening mechanism known in the art, for example a threaded connection, a bolted connection, or a latched connection. A modular rostral posterior component may then be attached by a similar fastening mechanism to the bridge to complete the rostral portion of the arthroplasty half. Likewise, the caudal components of the inferior arthroplasty half may be modular.

The arthroplasty halves 102, 104 may be formed of any suitable biocompatible material including metals such as cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. The various components comprising the arthroplasty halves 102, 104 may be formed of different materials thus permitting metal on metal, metal on ceramic, metal on polymer, ceramic on ceramic, ceramic on polymer, or polymer on polymer constructions.

Bone contacting surfaces of the arthroplasty halves 102, 104 may include features or coatings which enhance the fixation of the implanted prosthesis. For example, the surfaces may be roughened such as by chemical etching, bead-blasting, sanding, grinding, serrating, and/or diamond-cutting. All or a portion of the bone contacting surfaces of the arthroplasty halves 102, 104 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes, ridges, and/or other surface textures.

The artificial intervertebral joint 100 may be installed between the vertebrae 7, 9 as will be described below using a bilateral delivery. Generally, the artificial intervertebral joint 100 may be implanted into a body using a posterior transforaminal approach similar to the known TLIF or PLIF procedures. PLIF approaches are generally more medial and rely on more retraction of the traversing root and dura to access the vertebral interspace. The space between these structures is known as Kambin's triangle. TLIF approaches are typically more oblique, requiring less retraction of the exiting root, and less epidural bleeding with less retraction of the traversing structures. It is also possible to access the interspace using a far lateral approach, above the position of the exiting nerve root and outside of Kambin's triangle. In some instances it is possible to access the interspace via the far lateral without resecting the facets. Furthermore, a direct lateral approach through the psoas is known. This approach avoids the posterior neural elements completely. Embodiments of the current invention are anticipate that could utilize any of these common approaches.

According to at least one of these approaches, an incision, such as a midline incision, may be made in the patient's back and some or all of the affected disc and surrounding tissue may be removed via the foramina. The superior endplate surface of the vertebra 9 may be milled, rasped, or otherwise resected to match the profile of the caudal anterior bone contacting surface 112a, to normalize stress distributions on the superior endplate surface of the vertebra 9, and/or to provide initial fixation prior to bone ingrowth. The preparation of the endplate of vertebra 9 may result in a flattened surface or in surface contours such as pockets, grooves, or other contours that may match corresponding features on the bone contacting surface 112a. The inferior endplate of the vertebra 7 may be similarly prepared to receive the rostral anterior joint component 106 to the extent allowed by the exiting nerve root and the dorsal root ganglia. Depending on whether any of the facet joints are being replaced or supplemented, the natural facet joints of vertebrae 7, 9 may be trimmed to make room for the posterior components 108, 114.

The superior arthroplasty half 102 of the artificial intervertebral joint 100 may then be inserted piecewise through, for example, a right transforaminal exposure. That is, the rostral anterior component 106 may be inserted through the foramina and is placed in the appropriate intervertebral disc space between the generally cylindrical bodies 7a, 9a. The anterior components 106 may be delivered along a curved or angled path similar to that used with other types of TLIF grafts. The articulating joint replacement assembly 104 of the artificial intervertebral joint 100 may then be inserted piecewise through a contralateral exposure, for example, a left transforaminal exposure. That is, the caudal anterior joint component 112 may be inserted through the contralateral foramina and is placed in the appropriate intervertebral disc space between the generally cylindrical bodies 7a, 9a. The caudal anterior joint component 112 may also be delivered along a curved or angled path similar to that used with other TLIF grafts or may be delivered along any other path that accommodates the shape of the components. It is understood that the arthroplasty halves may be configured such that the inferior half may be inserted from right exposure, and the superior half may be inserted from the left exposure.

Within the intervertebral disc space, the anterior components 106, 112 may be positioned such that the anterior socket 122 is pivotally engaged with the curved protrusion 118 to form a ball and socket style joint. The center of rotation 124 is thus fixed within the intervertebral disc space or, depending upon the amount of resection performed, within the inferior generally cylindrical body 9a. This location for the center of rotation 124 may generally be within the natural center of rotation for the joint formed by the adjacent vertebrae 7, 9.

Outside the intervertebral disc space and posteriorly of a vertebral canal formed by the adjacent vertebrae 7, 9, the posterior arm 108 may engaged with the posterior arm 114 such that the curved tabs 128, 130 are inserted into the curved slots 132, 134. The posterior arms 108, 114 may extend across the axis 125 such that curved tab 128 engages curved slot 132 to form a posterior joint on one side of the axis 125, and the curved tab 130 engages with the curved slot 134 to form a second posterior joint on the opposite side of the axis 125. These posterior components 108, 114 may replace or supplement the function of the natural facet joints and may be useful in treating arthritis and degenerative changes of the facet joints.

Installation of the posterior arms 108, 114 may involve resection of at least a portion of either or both of the spinous process 7b and/or the spinous process extending from the vertebra 9. The notch 140 may provide space to accommodate the whole or the resected portion of the spinous process of vertebra 9. In an alternative embodiment, the posterior arms may connect through or attach to a spinous process.

The bridges 110, 116 may extend posteriorly from the anterior joint components 106, 112, respectively and posteriorly from the intervertebral disc space. In addition to joining the anterior and posterior components, the bridges 110, 116 may serve to prevent subsidence. By crossing onto either the pedicle (for caudal bridges 116) or the posterior wall of the apophyseal ring of vertebra 7 (for rostral bridges 110), greater surface area is created and bone subsidence may be reduced. Once installed, the arthroplasty halves 102, 104 may surround a cross-section of the vertebral canal between the superior and inferior vertebrae 7, 9.

After installation, the superior arthroplasty half 102 and the inferior arthroplasty half 104 may be secured to vertebrae 7, 9. The screw 152 may be inserted through the connection component 150 and into the generally cylindrical body 7a. The screw 156 may be inserted through the connection component 154 and may be affixed extrapedicularly to the vertebra 9. For example, the screw 156 may pass through a lateral wall of the pedicle to achieve strong cortical fixation. It is understood that the screws may be implanted either after the entire arthroplasty half has been implanted or after each of the rostral and caudal component has been implanted. As described above, the connection components and the screws may be omitted altogether.

As installed, the anterior ball and socket type joint created by the anterior joint components 106, 112 may be relatively stable and self-centering. While the anterior articulating surfaces 118, 122 pivot about the center of rotation 124, the tabs 128, 130 may slide within the slots 132, 134, respectively. As they slide through the slots, the tabs 128, 130 may move or revolve on an arc-shaped path about the same center of rotation 124. Because the curved tabs 128, 130 and the curved slots 132, 134, share a common center of rotation 124 with the anterior articulating surfaces 118, 122, the anterior ball and socket joint may enjoy a full range of motion, subject to the limits provided by the posterior arms 108, 114. For example, the curved tabs 128, 130 positioned within the slots 132, 134 may serve to resist shear forces, particularly anterior-posterior forces, preventing disarticulation of the ball and socket joint formed by curved protrusion 118 and the anterior socket 122. Lateral translation and rotational motion of the rostral anterior component 106 relative to the caudal anterior component 112 may also be limited by the posterior arms 108, 114. For example, limiting walls 136, 138 may act as stops for curved tabs 128, 130, limiting movement of the tabs within the slots 132, 134, respectively, and thus limiting the rotation of the tabs about the center of rotation 124. Flexion-extension motion in the anterior ball and socket joint may be permitted as the curved tabs 128, 130 are permitted to lift within the slots 132, 134, respectively. The curved tabs 128, 130 may even be allowed to decouple from the slots 132, 134, respectively, to permit greater flexion motions. Under certain conditions, the anterior joint components 106, 112 may become disconnected and/or the tabs 128, 130 may become decoupled from slots 132, 134, to permit additional degrees of freedom and coupled motions beyond those permitted by the fully engaged anterior and posterior joints. The self-centering nature of the anterior joint may encourage reconnection and alignment after decoupling occurs.

In general, a simple, anteriorly located ball and socket joint which is tightly constrained with each component having the same or similar radii of curvature may allow flexion-extension, lateral bending, and torsion motions while resisting shear forces and limiting translation. By adding an additional highly constrained ball and socket joint to the posterior components, an additional degree of freedom may be limited, such as torsion. Additional joints may further limit degrees of freedom of motion. If the anterior or posterior joints are permitted to disconnect or disarticulate additional degrees of freedom may be permitted as described above. Changing the shape of or clearance between the ball and socket components will also permit additional degrees of motion.

In an alternative embodiment, the artificial intervertebral joint described above may further include a rostral keel extending from the rostral anterior component and/or a caudal keel extending from the caudal anterior joint component and along the caudal bridge. The rostral keel may engage the inferior endplate of the vertebral body 7a, and the caudal keel may engage the superior endplate of the vertebral body 9a and a superior face of a pedicle of vertebra 9. It is understood that the inferior endplate of the body 7a may be milled or otherwise prepared to receive the rostral keel. Likewise, the superior endplate of the body 9a and the pedicle of vertebra 9 may be milled, chiseled, or otherwise prepared to create a channel for receiving the caudal keel. The keels may help to connect to the bone and limit movement of the arthroplasty half to the desired degrees to freedom. The keels may have an angled or semi-cylindrical cross section. It is understood that more than one keel may be used on any given component.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,”and “right,” are for illustrative purposes only and can be varied within the scope of the disclosure. In the claims, means-plus-function clauses are intended to cover the structures described herein as performing the recited function and not only structural equivalents, but also equivalent structures.

1. An artificial spinal joint for creating at least a portion of a coupling between a superior vertebra and an inferior vertebra comprising:
an inferior arthroplasty half comprising an inferior articulating component for placement in an intervertebral disc space between the superior and inferior vertebrae, a first posterior arm, and a first bridge component coupled between the inferior articulating component and the first posterior arm; and
a superior arthroplasty half comprising a superior articulating component for placement in an intervertebral disc space between the superior and inferior vertebrae, a second posterior arm, and a second bridge component coupled between the superior articulating component and the second posterior arm,
wherein the first posterior arm and the second posterior arm cross an anterior-posterior axis defined centrally through and extending from the intervertebral disc space.

2. The artificial spinal joint of claim 1 wherein inferior and superior arthroplasty halves surround a cross section of a vertebral canal defined between the superior and inferior vertebrae.

3. The artificial spinal joint of claim 1 wherein the second posterior arm is configured to engage the first posterior arm.

4. The artificial spinal joint of claim 3 wherein the second posterior arm is configured to engage the first posterior arm in at least a first joint and a second joint.

5. The artificial spinal joint of claim 4 wherein the first and second joints are on opposite sides of the anterior-posterior axis.

6. The artificial spinal joint of claim 4 wherein the first joint comprises a first slot in the first posterior arm adapted to engage a first tab of the second posterior arm.

7. The artificial spinal joint of claim 6 wherein the second joint comprises a second slot in the first posterior arm adapted to engage a second tab of the second posterior arm.

8. The artificial spinal joint of claim 1 wherein the inferior articulating component comprises a curved protrusion and the superior articulating component comprises a socket adapted to receive the curved protrusion.

9. The artificial spinal joint of claim 1 wherein the inferior articulating component is pivotally engaged with the superior articulating component.

10. The artificial spinal joint of claim 1 wherein the first bridge component extends posteriorly from the inferior articulating component and outwardly of the intervertebral disc space.

11. The artificial spinal joint of claim 1 wherein the second bridge component extends posteriorly from the superior articulating component and outwardly of the intervertebral disc space.

12. The artificial spinal joint of claim 1 wherein the second bridge component comprises a jog adapted to permit passage of a neural element.

13. The artificial spinal joint of claim 1 further comprising a bone fastener for attaching the artificial spinal joint to either the superior vertebra or the inferior vertebra.

14. The artificial spinal joint of claim 13 wherein the superior arthroplasty half further comprises a connection component adapted to receive the bone fastener.

15. The artificial spinal joint of claim 14 wherein the bone fastener is a bone screw and the connection component is further adapted to direct the received bone screw into a generally cylindrical body portion of the superior vertebra.

16. The artificial spinal joint of claim 13 wherein the inferior arthroplasty half further comprises a connection component adapted to receive the bone fastener.

17. The artificial spinal joint of claim 14 wherein the bone fastener is a bone screw and the connection component is further adapted to direct the received bone screw for extrapedicular connection to the inferior vertebra.

18. The artificial spinal joint of claim 1 wherein the inferior articulating component is sized for insertion through Kambin's triangle.

19. The artificial spinal joint of claim 1 wherein the inferior arthroplasty half further comprises an extension surface extending anteriorly of the inferior articulating component.

20. The artificial spinal joint of claim 1 wherein the first posterior arm comprises a notch adapted to receive at least a portion of a spinous process.

21. The artificial spinal joint of claim 1 wherein the first bridge component is at least a portion of an artificial pedicle.

22. A method of implanting an artificial spinal joint between superior and inferior vertebrae, the method comprising:
creating a first exposure through a patient's back to access an intervertebral space;
creating a second exposure through the patient's back to access the intervertebral space;
delivering a first articulating portion of the artificial spinal joint to the intervertebral space along a first path through the first exposure;
delivering a second articulating portion of the artificial spinal joint to the intervertebral space along a second path through the second exposure;
engaging the first and second articulating assembly portions to form an intervertebral joint centered about an anterior-posterior axis defined through and extending from the center of the intervertebral disc space; and
positioning first and second posterior arms of the artificial spinal joint outside of the intervertebral space and across the anterior-posterior axis.

23. The method of claim 22 wherein the step of positioning the first and second posterior arms comprises positioning the first and second posterior arms in movable engagement.

24. The method of claim 23 wherein the step of positioning the first and second posterior arms further includes restricting displacement of the first posterior arm with respect to the second posterior arm to restrict rotational movement in the intervertebral joint.

25. The method of claim 23 wherein the step of positioning the first and second posterior arms further includes restricting displacement of the first posterior arm with respect to the second posterior arm to restrict generally anterior-posterior shear motion in the intervertebral joint.

26. The method of claim 22 wherein the intervertebral joint is a ball and socket type joint.

27. The method of claim 21 wherein the first posterior arm is integrally formed with the first articulating assembly portion and the second posterior arm is integrally formed with the second articulating assembly portion.

28. The method of claim 22 further comprising:
removing at least a portion of a spinous process of either the superior or inferior vertebra.

29. The method of claim 22 further comprising:
engaging at least a portion of a spinous process with a recessed portion of the first posterior arm.

30. The method of claim 22 wherein the first path is curved.

31. The method of claim 22 wherein the second path is contralateral to the first path.

32. A system for creating a coupling between a superior vertebra and an inferior vertebra, the system comprising:
rostral and caudal anterior articulating components pivotally engaged about a center of rotation, wherein the rostral and caudal anterior articulating components and the center of rotation are adapted for location within an intervertebral disc space between the superior and inferior vertebrae; and
rostral and caudal posterior arms adapted for positioning outside of the intervertebral disc space and slidably engaged to move about the center of rotation,
wherein the rostral anterior articulating component is adapted for implantation through a first approach into the intervertebral disc space and the caudal articulating component is adapted for implantation through a contralateral approach into the intervertebral disc space.

33. The system of claim 32 wherein the rostral posterior arm comprises at least one curved tab having a center of curvature located at the center of rotation.

34. The system of claim 32 wherein the caudal posterior arm comprises at least one curved slot adapted to receive the at least one curved tab.

35. The system of claim 34 wherein the at least one curved slot includes a first curved slot and a second curved slot and the at least one curved tab includes a first curved tab adapted to slide within the first curved slot and a second curved tab adapted to slide within the second curved slot.

36. The system of claim 35 wherein the first and second curved tabs are adapted to move about the center of rotation while sliding in the first and second curved slots, respectively.

37. The system of claim 32 wherein the rostral posterior arm is rigidly connected to the rostral anterior articulating component and the caudal posterior arm is rigidly connected to the caudal anterior articulating component.

38. The system of claim 37 further comprising:
a rostral bridge component rigidly connecting the rostral anterior articulating component and the rostral posterior arm and
a caudal bridge component rigidly connecting the caudal anterior articulating component and the caudal posterior arm.

39. A system for creating at least a portion of a coupling between a superior vertebra and an inferior vertebra comprising:
a first means adapted for articulation in an intervertebral disc space between the superior and inferior vertebrae;
a second means coupled to the first means and adapted for articulation posteriorly of the intervertebral disc space; and
a third means coupled to the first means and adapted for articulation posteriorly of the intervertebral disc space,
wherein the second and third means are interconnected for articulation posteriorly of the intervertebral disc space and wherein the second and third means both cross an anterior-posterior axis defined centrally through and extending from the intervertebral disc space.
